(19) United States
(12) Patent Application Publication
(10) Pub. No.: US 2025/0312528 A1
(43) Pub. Date: Oct. 9, 2025

(54) Automatic Anesthetic Buffering System And Method

(71) Applicant: Guy M. Ingrim, Anchorage, AK (US)

(72) Inventor: Guy M. Ingrim, Anchorage, AK (US)

(21) Appl. No.: 19/169,943

(22) Filed: Apr. 3, 2025

Related U.S. Application Data
(60) Provisional application No. 63/573,583, filed on Apr. 3, 2024.

(51) Int. Cl. A61M 5/14; A61M 5/24

(52) U.S. Cl. CPC A61M 5/1409; A61M 5/2429; A61M 5/2466; A61M 2005/2474; A61M 2202/0007; A61M 2202/048

(57) ABSTRACT

An automatic anesthetic buffering system is provided. The system comprises a carpule having a body that defines an internal chamber, a forward end configured to be pierced by a needle, and a rear end configured to receive a plunger actuation force. A bladder assembly is positioned within the carpule and includes a collapsible bladder containing a buffer solution. An anesthetic is positioned toward the rear end of the carpule. Actuation of the plunger causes the bladder to collapse and discharge the buffer solution through the needle prior to delivery of the anesthetic. In one embodiment, the bladder assembly includes a bladder retention spring, a radial spring clip, and a bladder cushion. The system enables in situ mixing of the buffer and anesthetic solutions immediately prior to or during injection, reducing injection pain and improving anesthetic onset.

### CROSS-REFERENCE TO RELATED APPLICATIONS

This application claims the benefit of priority to pending U.S. Provisional Patent Application No. 63/573,583, filed on Apr. 3, 2024, the entire contents of which are hereby incorporated by reference in their entirety

### BACKGROUND OF THE INVENTION

The present invention relates to anesthetic buffering systems and methods. More particularly, the invention provides a system and method for automatically buffering an anesthetic solution using a buffer cartridge or bladder contained within a carpule. The buffer solution is configured to mix with the anesthetic solution at the time of administration, reducing pain and improving anesthetic performance without requiring separate preparation steps by the practitioner.

Local anesthetics are commonly used in dental and medical procedures to numb tissue and improve patient comfort. In practice, local anesthetic is injected into the patient's soft tissue near the target area, where it inhibits nerve signals and blocks pain. These anesthetics are typically supplied in glass carpules and may include additives such as epinephrine to prolong anesthetic duration by constricting blood vessels at the injection site.

While epinephrine helps to localize the anesthetic effect, it also decreases the pH of the solution, making it more acidic. This acidity can cause a stinging sensation during injection and reduce the proportion of the anesthetic that is in its active, non-ionized form. As a result, onset of the anesthesia may be delayed, and additional injections may be required, increasing cost and patient discomfort.

Buffering agents can be used to neutralize the acidity of the anesthetic solution. Buffering raises the pH, reduces injection pain, and improves onset time. However, manufacturers cannot pre-buffer anesthetic solutions during production, as doing so would drastically reduce shelf life. Therefore, buffered anesthetics must be prepared at the point of care.

In current clinical settings, buffering can be performed manually. A typical process involves removing a portion of the anesthetic from the carpule, drawing buffer solution from a separate container into a second syringe, and transferring it into the anesthetic carpule. This manual technique is inefficient, imprecise, and wasteful. It also increases the risk of contamination and must be repeated for each use. Other systems involve specialized carpules or external buffer cartridges, which may be costly or mechanically complex, and may still require additional steps prior to use.

In some systems, internal piercing mechanisms or needles are incorporated into the carpule to release buffer from an internal cartridge. While potentially effective, these systems introduce additional manufacturing cost and failure points due to the complexity of internal needles and precision components.

Recognizing the inefficiencies in current practices, the present invention introduces an improved system for automatically mixing a buffer solution with an anesthetic solution within a carpule. The system includes a collapsible buffer cartridge or bladder positioned toward the forward end of the carpule, and an anesthetic solution positioned toward the rear end. In some embodiments, the buffer bladder is supported by a restraint assembly comprising a bladder retention spring, a radial spring clip, and a bladder cushion. In other embodiments, a thermoplastic rubber anchor replaces the clip and cushion while securing the bladder in position. When the carpule is actuated by a syringe plunger, the buffer solution is discharged first through a needle, followed by the anesthetic, enabling consistent in situ buffering during injection.

In light of the devices disclosed in the known art, it is submitted that the present invention substantially diverges in design elements and methods from the known art and consequently it is clear that there is a need in the art for an improvement to anesthetic buffering systems and methods. In this regard the instant invention substantially fulfills these needs.

### SUMMARY OF THE INVENTION

In view of the limitations associated with existing anesthetic buffering systems and methods, the present invention provides an automatic anesthetic buffering system configured to deliver a buffer solution and an anesthetic solution sequentially from a single carpule. The system enables in situ mixing of the two solutions at the time of administration, improving delivery efficiency, reducing injection pain, and accelerating anesthetic onset.

In one embodiment, the system comprises a carpule having a body that defines an internal chamber, a forward end configured to be pierced by a needle, and a rear end configured to receive an actuation force from a syringe plunger. A bladder assembly is positioned within the carpule and includes a collapsible bladder containing a buffer solution. An anesthetic solution is positioned toward the rear of the carpule. Actuation of the plunger causes the bladder to collapse and discharge the buffer solution through the needle prior to delivery of the anesthetic.

In one embodiment, the bladder assembly includes a bladder retention spring, a radial spring clip, and a bladder cushion to support the bladder and transmit force during actuation. In another embodiment, the bladder assembly includes a thermoplastic rubber anchor configured to receive a flat portion of the bladder, in combination with a retention spring.

It is an objective of the invention to reduce the need for manual buffering procedures and conserve anesthetic material by eliminating waste. The system enables rapid and consistent buffering directly within the carpule and is compatible with standard syringe formats.

It is another objective to provide a system that can be integrated during manufacturing or retrofit into existing carpules, thereby offering flexibility for deployment in various clinical workflows.

It is therefore an object of the present invention to provide new and improved automatic anesthetic buffering systems and methods that have all of the advantages of the known art and none of the disadvantages.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings.

FIG. 1 is a perspective view of an embodiment of an automatic anesthetic buffering system.

FIG. 2 is an exploded view of a bladder assembly of an embodiment of the automatic anesthetic buffering system.

FIG. 3 is a perspective view of a bladder assembly of an embodiment of the automatic anesthetic buffering system.

FIG. 4 is a side view of an embodiment of a bladder of an embodiment of the automatic anesthetic buffering system.

FIG. 5 is a cross-sectional view of an embodiment of the automatic anesthetic buffering system taken along line 5-5 of FIG. 1.

FIG. 6 is an exploded view of a syringe system including a stylus, a syringe, and a carpule of an embodiment of the automatic anesthetic buffering system.

FIG. 7 is a perspective view of a second embodiment of the bladder assembly of the automatic anesthetic buffering system.

FIG. 8 is an exploded view of the second embodiment of the bladder assembly of the automatic anesthetic buffering system.

FIG. 9 is an end view of a rubber anchor of the second embodiment of the bladder assembly of the automatic anesthetic buffering system.

### DETAILED DESCRIPTION OF THE INVENTION

Reference is made herein to the attached drawings. For the purpose of presenting a brief and clear description of the present invention, the preferred embodiment will be discussed as used for automatically mixing a buffer solution with an anesthetic solution at the same time that the anesthetic solution is administered to the patient. The figures are intended for representative purposes only and should not be considered to be limiting in any respect.

Reference will now be made in detail to the exemplary embodiment(s) of the invention. References to “one embodiment,” “at least one embodiment,” “an embodiment,” “one example,” “an example,” “for example,” and so on indicate that the embodiment(s) or example(s) may include a feature, structure, characteristic, property, element, or limitation but that not every embodiment or example necessarily includes that feature, structure, characteristic, property, element, or limitation. Further, repeated use of the phrase “in an embodiment” does not necessarily refer to the same embodiment.

As used herein, a “carpule” or “carpoule” refers to a cylindrical container typically composed of glass or polymer, adapted to hold one or more injectable fluids such as an anesthetic or buffer solution. The carpule may be pre-filled or filled during a procedural step and is configured to be received within a syringe for administration. A “syringe”, as used herein, refers to a handheld device comprising a barrel and a plunger or drive rod. The syringe is configured to receive and actuate a carpule by applying axial pressure to a movable element at its rear end, thereby expelling the fluid contents through an attached needle.

The term “bidirectional needle” refers to a hollow needle having a proximal end and a distal end. The proximal end is configured to pierce one or more fluid-retaining components within the carpule, such as a septum or bladder, while the distal end is configured to deliver the contents of the carpule into the soft tissue of a patient. The bidirectional needle establishes a continuous fluid path from the interior of the carpule to the injection site.

A “bladder”, as used herein, refers to a collapsible, deformable reservoir containing a buffer solution. The bladder is configured to deform in response to axial compression, thereby discharging its contents through the carpule's forward end. In some embodiments, the bladder includes structural features such as a convoluted wall and flat panel to aid controlled deformation.

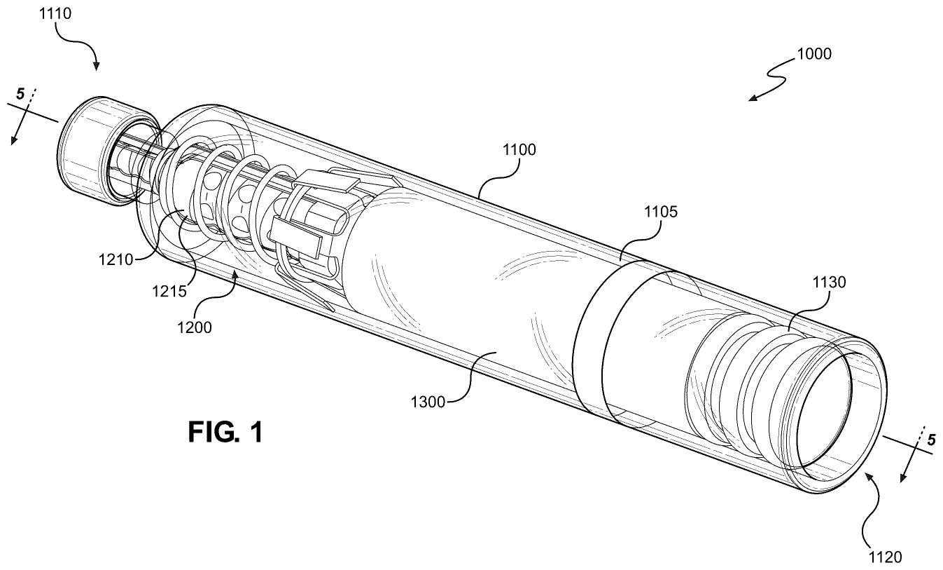

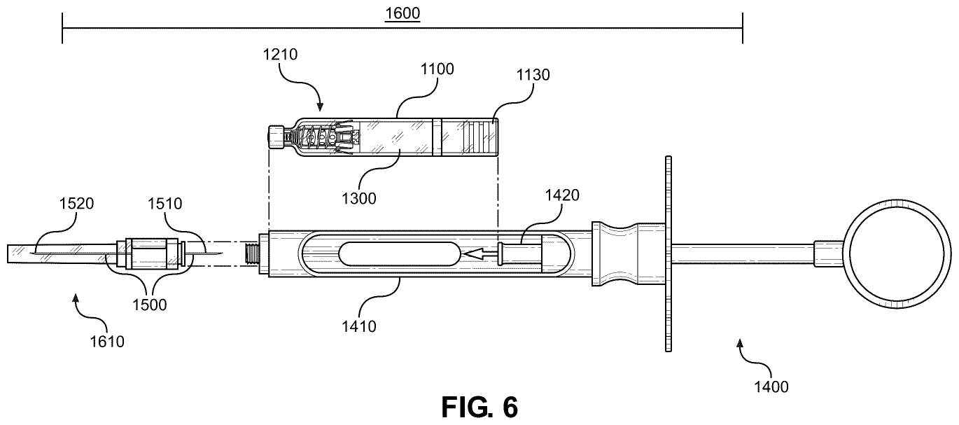

Referring now to FIG. 1, an embodiment of an automatic anesthetic buffering system 1000 is shown in a fully assembled state. The system 1000 includes a carpule 1100, a bladder assembly 1200, and an anesthetic volume 1300. The carpule 1100 is configured to be received within a syringe 1400 and to interface with a bidirectional needle (shown in FIG. 6, 1500). The carpule 1100 defines an elongated, substantially cylindrical body having an internal chamber 1105. The carpule includes a forward end 1110 and a rear end 1120. The forward end 1110 terminates in a pierceable barrier or septum (not shown in this view), which is configured to be punctured by the proximal end of the bidirectional needle. The rear end 1120 includes a sealing plunger 1130, typically formed of rubber or elastomeric material, which is axially movable and configured to transmit force from the syringe plunger. The carpule 1100 may be constructed from glass or polymer, and in one embodiment includes a metal ferrule and crimp ring to secure the septum in place.

A bladder assembly 1200 is disposed toward the forward end 1110 of the carpule 1100. The bladder assembly 1200 includes a collapsible bladder 1210 that defines an internal reservoir filled with a buffer solution 1215. In the illustrated embodiment, the bladder 1210 is positioned concentrically within the internal chamber 1105 and is oriented such that it discharges toward the forward end 1110. The bladder 1210 may have a convoluted or pleated wall structure to facilitate controlled collapse, and may include a longitudinal flat panel 1212 to guide deformation along a preferred axis.

Positioned behind the bladder assembly 1200 is an anesthetic volume 1300. In one embodiment, the anesthetic volume is a liquid anesthetic solution pre-loaded or aspirated into the carpule 1100 during assembly. The anesthetic volume 1300 is located between the bladder assembly 1200 and the sealing plunger 1130, such that the anesthetic is dispensed only after the buffer solution 1215 has been discharged.

A syringe (shown in FIG. 6) is configured to receive the carpule 1100 within a barrel or receptacle 1410. The syringe includes a drive plunger 1420, which is actuated by the user to apply an axial force to the carpule's sealing plunger 1130. A bidirectional needle (shown in FIG. 6) is secured to a forward end of the syringe. Upon assembly, the proximal end of the needle pierces the carpule's septum and the forward portion of the bladder 1210. The distal end 1520 of the needle is configured for subcutaneous or soft tissue insertion into a patient.

In operation, forward actuation of the drive plunger 1420 causes the sealing plunger 1130 to move axially within the carpule 1100. This compresses the bladder 1210, causing it to collapse and discharge the buffer solution 1215 through the bidirectional needle. Continued plunger actuation then forces the anesthetic solution 1300 through the same fluid path, resulting in a sequential injection where the buffer precedes the anesthetic. This configuration improves pH normalization at the injection site, reduces perceived injection pain, and accelerates the onset of anesthesia.

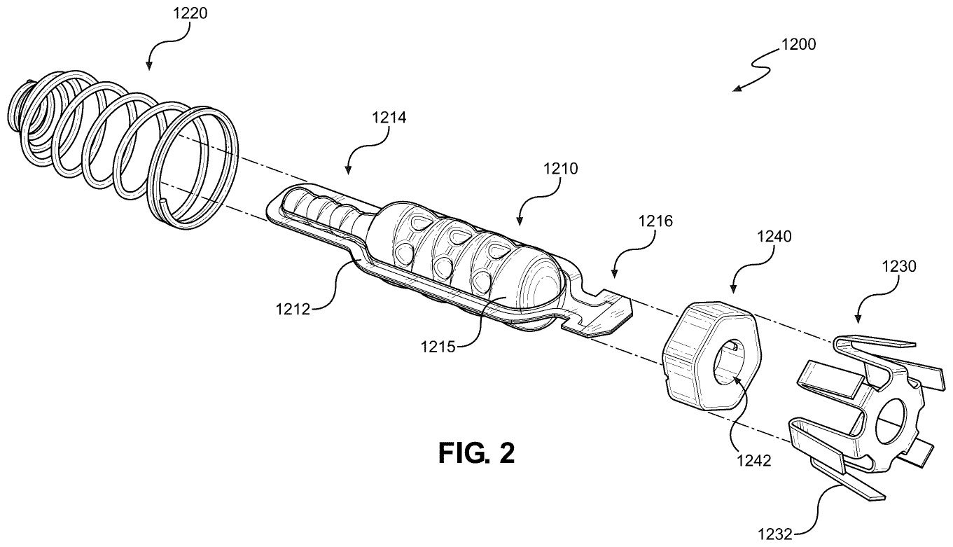

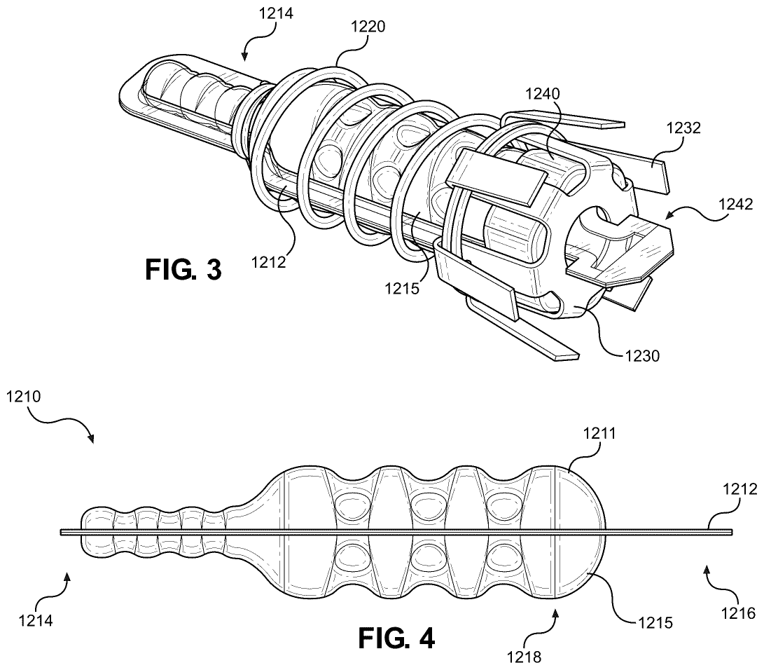

Referring now to FIGS. 2 and 3, a buffer assembly 1200 of an embodiment of the automatic anesthetic buffering system 1000 is shown in both exploded (FIG. 2) and assembled (FIG. 3) views. The buffer assembly 1200 is configured for placement within the carpule 1100, as previously described with respect to FIG. 1. The buffer assembly 1200 includes a bladder 1210, a bladder retention spring 1220, a spring clip 1230, and a bladder cushion 1240.

The bladder 1210 defines a closed reservoir that contains a buffer solution 1215. The bladder 1210 is generally cylindrical and may include a convoluted wall structure formed by alternating ridges and valleys to promote predictable, uniform collapse under axial compression. A flat longitudinal panel 1212 runs along one side of the bladder, configured to guide folding and prevent twisting during collapse. The first end 1214 of the bladder includes a pierceable septum or seal, which is positioned to be pierced by the proximal end 1510 of the bidirectional needle 1500 when installed. The second end 1216 of the bladder may include a flat anchor tab or flange to assist with positioning and alignment within the assembly.

Surrounding a portion of the bladder 1210 is the bladder retention spring 1220. The spring 1220 is a helical or coil spring configured to apply an axial force against the bladder 1210 when compressed. The spring 1220 is sized to fit concentrically around the midsection of the bladder and is positioned between the bladder cushion 1240 and the syringe plunger interface.

The spring clip 1230 is positioned at the forward end of the buffer assembly 1200. In the exploded view of FIG. 2, the spring clip 1230 includes a plurality of radially spaced arms 1232 that extend circumferentially around an inner aperture. The spring clip is formed of resilient material, such as stainless steel, and is configured to flex radially during insertion into the carpule 1100. When fully inserted, the clip 1230 expands to engage the inner wall of the carpule, forming an interference fit that resists rearward movement.

The bladder cushion 1240 is positioned between the spring clip 1230 and the bladder 1210. The cushion 1240 is formed of a compliant elastomer such as silicone rubber and includes a central bore 1242 configured to receive the second end 1216 of the bladder. The cushion 1240 serves to distribute compressive force from the spring and syringe plunger evenly across the base of the bladder. In the assembled view of FIG. 3, the bladder cushion is captured between the spring clip 1230 and the retention spring 1220, with the bladder 1210 partially extending through its central bore.

During assembly of the buffer assembly 1200, the bladder 1210 is first inserted into the central cavity of the bladder cushion 1240 such that the second end 1216 is received in the bore 1242. The spring clip 1230 is aligned to the forward face of the bladder cushion, and the bladder retention spring 1220 is positioned concentrically around the bladder, extending rearward. The spring 1220 is then partially compressed to hold all elements together in axial alignment. The fully assembled buffer assembly 1200, as shown in FIG. 3, may then be inserted into the carpule 1100 from the rear end 1120, prior to addition of the anesthetic solution 1300.

When installed, the spring clip 1230 resists rearward movement by engaging the inner wall of the carpule. The retention spring 1220 stores axial potential energy, which, when actuated by the syringe plunger, is transmitted through the bladder cushion 1240 to the bladder 1210. This force causes the bladder to collapse and discharge the buffer solution 1215 through the pierced septum at the forward end 1214.

Referring now to FIG. 4, a side view of the bladder 1210 is shown. The bladder 1210 is an elongate, deformable reservoir that defines an interior chamber 1218 pre-filled with a buffer solution 1215. In the shown embodiment, the bladder is generally cylindrical but is configured to control deformation during actuation. For example, the outer wall 1211 of the bladder comprises a series of convoluted ridges and troughs, forming a pleated or accordion-like structure. This geometry promotes controlled axial collapse while resisting radial deformation or twisting.

In the illustrated embodiment, the bladder 1210 also includes a flat longitudinal panel 1212 that extends along one side of the bladder and lies in a plane parallel to the bladder's central longitudinal axis. The panel 1212 provides a predictable fold axis during compression and prevents the bladder from rotating or buckling off-axis. At the first (forward) end 1214, the bladder includes a septum or pierceable seal, formed of rubber or elastomer, configured to be punctured by the proximal end 1510 of the bidirectional needle 1500 when the carpule is assembled for use.

The second (rear) end 1216 of the bladder includes a generally flat flange or tab, which serves as a physical interface with the bladder cushion 1240 and also anchors the bladder in axial alignment within the restraint assembly. In one embodiment, the tab includes an embedded seam or peripheral sealing feature that aids in positioning during assembly.

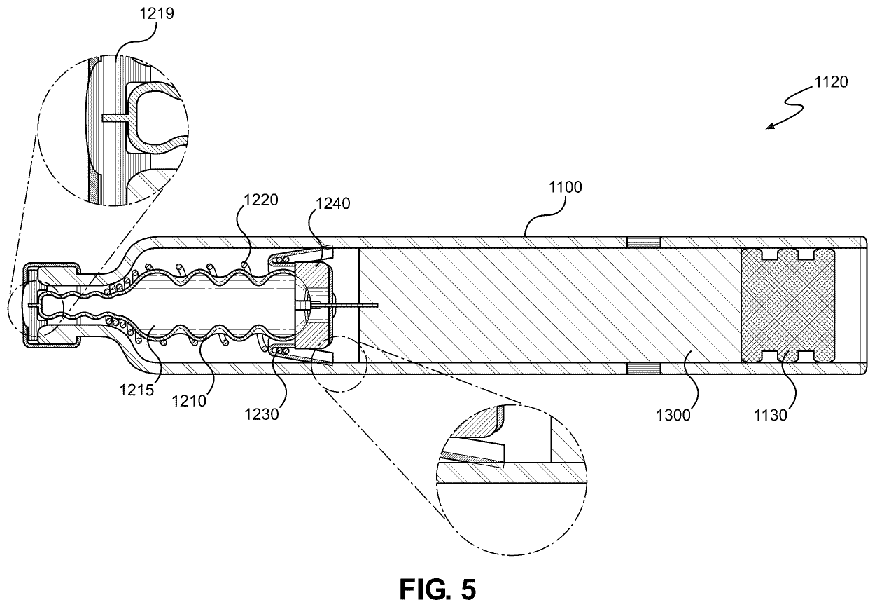

Referring now to FIG. 5, a cross-sectional view of the fully assembled system 1000 is shown, taken along line 5-5 of FIG. 1. The bladder 1210 is shown positioned within the forward end of the carpule 1100, with the bladder cushion 1240, spring clip 1230, and retention spring 1220 concentrically arranged around it. The proximal end of the bidirectional needle extends through the forward septum 1219 of the carpule 1110 and has pierced the forward septum 1219 of the bladder, forming a continuous fluid path.

In this state, the system 1000 is primed for activation. The anesthetic solution 1300 is located behind the bladder assembly 1200, with the sealing plunger 1130 positioned at the rear end 1120 of the carpule. When the syringe plunger 1420 (see FIG. 6) is actuated, the plunger 1130 moves forward, applying axial force to the bladder assembly 1200. The retention spring 1220 compresses, and the force is transmitted through the bladder cushion 1240 to the base of the bladder 1210. As a result, the bladder collapses and expels the buffer solution 1215 through the needle. Once the bladder is fully collapsed, continued actuation of the syringe plunger displaces the anesthetic solution 1300 through the same needle, delivering both fluids in a controlled, sequential manner.

Referring now to FIG. 6, an exploded view of an embodiment of a syringe-based delivery system is shown. The system 1600 includes a stylus 1610, a syringe 1400, and a carpule 1100, which are aligned along a common axis for insertion and engagement. The configuration shown in FIG. 6 illustrates the relationship between components during assembly and how the carpule 1100 is loaded into the syringe 1400 for use.

The stylus 1610 is a rigid, hollow component that includes a bidirectional needle 1500. The stylus 1610 may be formed from metal or polymer and includes an internal fluid passageway. The proximal end 1510 of the bidirectional needle 1500 extends rearward from the stylus and is configured to pierce the septum 1219 at the forward end 1110 of the carpule 1100, as well as the forward end of the bladder 1210. The distal end 1520 of the needle 1500 extends forward from the stylus 1610 and is configured for insertion into the patient's soft tissue.

The syringe 1400 includes a barrel 1410 that defines an internal cavity for receiving the carpule 1100. At the rear of the syringe 1400 is a drive plunger 1420, which is configured to translate axially within the barrel 1410. During use, the carpule 1100 is inserted into the syringe barrel 1410 with its rear end 1120 oriented toward the plunger 1420, and its forward end 1110 aligned with the stylus 1610.

As the carpule 1100 is inserted into the syringe 1400, the proximal end 1510 of the bidirectional needle 1500 punctures the carpule septum 1219 and then pierces the forward end of the bladder 1210. This establishes a continuous fluid pathway from the bladder's interior to the distal tip 1520 of the needle 1500.

Once assembled, actuation of the drive plunger 1420 applies an axial force to the rubber plunger 1130 at the rear of the carpule 1100. This force compresses the bladder assembly 1200, causing the bladder 1210 to collapse and discharge the buffer solution 1215 through the bidirectional needle 1500. Continued depression of the plunger 1420 then displaces the anesthetic solution 1300, which is delivered through the same needle 1500 into the patient's soft tissue.

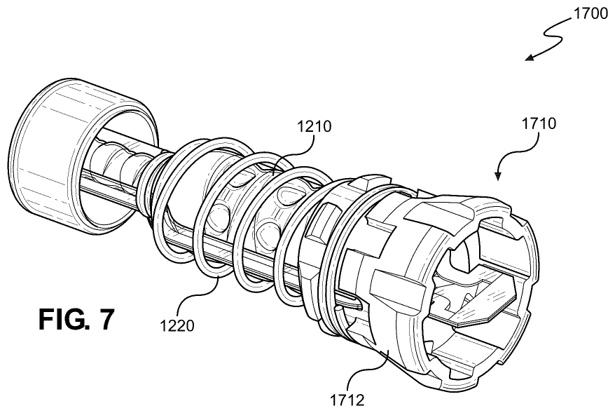

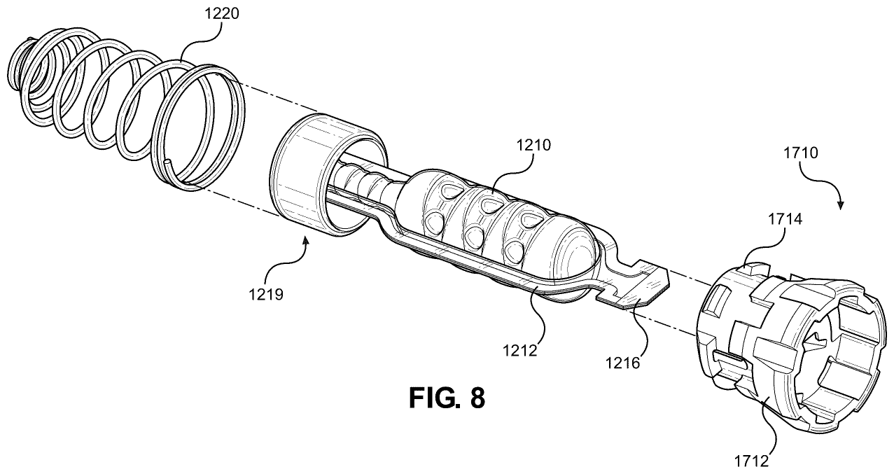

Referring now to FIGS. 7-9, there is shown a second embodiment of the bladder assembly 1700, which serves as an alternative to the bladder assembly previously described with reference to FIGS. 2-5. In this second embodiment, the radial spring clip and bladder cushion are replaced by a thermoplastic rubber anchor 1710, while the bladder 1210 and bladder retention spring 1220 are retained.

As shown in FIG. 7, the second embodiment of the bladder assembly 1700 includes a collapsible bladder 1210, a bladder retention spring 1220, and a rubber anchor 1710. These components are assembled within the carpule 1100 in a similar axial orientation as in the first embodiment. The bladder 1210 may be structurally identical to the one previously described, including the convoluted wall geometry, flat longitudinal panel, forward septum, and rear tab.

In the shown embodiment, the rubber anchor 1710 is a single-piece molded component composed of an elastomeric material such as thermoplastic rubber. The anchor 1710 includes a main body 1712 configured to seat against the inner wall of the carpule 1100 and to resist axial displacement under compression. The forward face 1714 of the anchor 1710 may include a contoured surface that interfaces with the carpule's forward interior geometry. The anchor is dimensioned to provide a friction fit or interference lock with the carpule interior, securing the bladder assembly 1700 in position during loading and actuation.

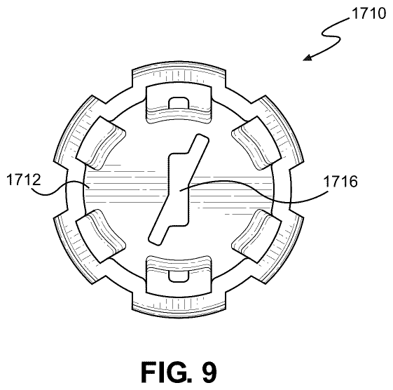

As shown more clearly in FIG. 8, the rubber anchor 1710 includes a slot 1716 formed through its main body. The slot 1716 is sized and shaped to receive the rear flat tab 1216 of the bladder 1210, thereby aligning the bladder longitudinally and rotationally within the anchor. The tab 1216 may engage a step or undercut within the slot to resist dislodgment during assembly or use. The anchor 1710 thus replaces the spring clip's mechanical arms and the bladder cushion's centering function with a single, integrated component.

In FIG. 9, an end view of the rubber anchor 1710 is shown, illustrating the slot 1716 and its positioning within the anchor's overall geometry. The slot is centered along the longitudinal axis of the anchor and opens rearward to receive the bladder tab 1216 during insertion. The anchor body 1712 may include one or more outer ridges or surface features configured to engage the carpule's interior and prevent axial or rotational movement.

During use, the bladder retention spring 1220 is positioned between the rear end 1120 of the carpule and the rear face 1718 of the rubber anchor 1710. When the syringe plunger 1420 is actuated, it compresses the spring 1220, which in turn applies force to the rubber anchor 1710 and thereby collapses the bladder 1210. The buffer solution 1215 is discharged through the pierced septum 1219, followed by the anesthetic solution 1300, as described in earlier sections.

In one embodiment, a method of administering a buffered anesthetic using an automatic anesthetic buffering system includes inserting a bladder assembly into a carpule body, the bladder assembly comprising a bladder containing a buffer solution. The bladder assembly includes a restraint structure such as a bladder retention spring with a spring clip and cushion, or a rubber anchor, configured to position and stabilize the bladder within the carpule. A volume of anesthetic is aspirated into the carpule body such that the anesthetic is positioned toward a rear end of the carpule. The carpule defines an internal chamber in which the buffer and anesthetic are retained in distinct regions, separated by the bladder assembly. A rubber plunger is inserted into the rear end of the carpule to seal the anesthetic volume and to receive actuation force from an external syringe plunger.

The method of administering a buffered anesthetic further comprises using inserting the carpule into a syringe having a stylus and a bidirectional needle, where the needle is positioned to pierce a septum at the forward end of the carpule and enter the bladder. In one embodiment, the stylus is configured to guide the bidirectional needle along a fixed trajectory for alignment with the bladder axis. Actuation of the syringe plunger applies a forward axial force to the rubber plunger, collapsing the bladder and discharging the buffer solution through the needle. The bladder deforms in a predictable manner under the constraint of the surrounding restraint, such as via a pleated wall and longitudinal flat panel. Continued actuation of the syringe plunger causes the anesthetic solution to be displaced through the same needle and delivered to the patient's soft tissue. The delivery pathway remains uninterrupted between the buffer discharge and anesthetic flow.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. An automatic anesthetic buffering system comprising:
a carpule comprising a body defining an internal chamber, a forward end configured to be pierced to dispense fluid via a needle, and a rear end configured to receive a plunger actuation force;
a bladder assembly positioned within the internal chamber, the bladder assembly comprising a collapsible bladder containing a buffer solution;
an anesthetic disposed towards the rear end;
wherein the bladder is configured to discharge the buffer solution through the forward end via the needle in response to a force applied at the rear end of the carpule.

2. The system of claim 1, further comprising a rubber plunger sealing the rear end of the carpule, the plunger configured to transmit actuation force to the bladder assembly.

3. The system of claim 1, wherein the forward end of the bladder includes a rubber septum configured to be pierced by the needle, the septum sealing the buffer solution within the bladder until actuation.

4. The system of claim 1, wherein the bladder comprises a convoluted wall structure with alternating wider and narrower segments adapted to collapse in a predictable manner during actuation.

5. The system of claim 4, wherein the bladder includes a flat panel disposed along a central longitudinal plane, the panel facilitating controlled folding during compression.

6. The system of claim 1, wherein the bladder comprises a housing defining a reservoir that tapers toward the forward end and includes a flat tab at a rear end.

7. The system of claim 1, further comprising a bladder restraint assembly disposed within the internal chamber, the restraint assembly comprising:
a bladder retention spring disposed around the bladder;
a radial spring clip; and
a bladder cushion positioned between the spring clip and the bladder.

8. The system of claim 7, wherein the spring clip comprises a plurality of radially spaced arms forming a compartment on a first side, the compartment configured to receive the bladder cushion.

9. The system of claim 8, wherein the spring clip includes an interference edge configured to engage an interior surface of the carpule body to resist axial displacement of the bladder assembly upon insertion.

10. The system of claim 9, wherein the spring clip is configured to travel freely during insertion and jam against the carpule wall when a forward force is applied.

11. The system of claim 7, wherein the bladder retention spring is configured to concentrically surround the bladder and apply a compressive force against the bladder cushion and spring clip during actuation.

12. The system of claim 7, wherein the bladder cushion comprises a silicone rubber body and includes a central bore sized to receive a portion of the rear end of the bladder.

13. The system of claim 7, wherein the bladder restraint assembly comprises a rubber anchor bladder assembly including a thermoplastic rubber anchor and a bladder retention spring.

14. The system of claim 13, wherein the thermoplastic rubber anchor comprises a slot sized to receive a flat portion of the bladder, the anchor configured to retain the bladder in a fixed orientation during actuation.

15. The system of claim 13, wherein the retention spring is configured to engage the rubber anchor and apply axial force to the bladder during actuation.

16. The system of claim 7, further comprising a syringe configured to receive the carpule, the syringe comprising:
a housing defining a chamber for receiving the carpule; and
a drive plunger configured to engage the rubber plunger at the rear end of the carpule to actuate delivery of the buffer solution and anesthetic.

17. A method of administering a buffered anesthetic using an automatic anesthetic buffering system, the method comprising:
inserting a bladder assembly into a carpule body, the bladder assembly comprising a bladder containing a buffer solution;
aspirating a volume of anesthetic into the carpule body such that the anesthetic is positioned toward a rear end of the carpule;
sealing the rear end of the carpule with a rubber plunger;
piercing a forward end of the bladder with a needle;
actuating the rubber plunger to apply a forward force;
collapsing the bladder in response to the forward force; and
dispensing the buffer solution through the needle prior to the anesthetic.

18. The method of claim 17, wherein the bladder is housed within a bladder restraint assembly comprising a bladder retention spring and either:
(a) a radial spring clip with a bladder cushion interposed between the clip and the bladder, or
(b) a thermoplastic rubber anchor having a slot that receives a flat portion of the bladder.

19. The method of claim 17, wherein the bladder comprises a convoluted wall structure and a longitudinally flat panel configured to collapse in a predictable manner under compression.
